(19) United States
(12) Patent Application Publication
(10) Pub. No.: US 2023/0181775 A1
(43) Pub. Date: Jun. 15, 2023

(54) METHODS OF USING AND CONVERTING RECOVERED RADIUM

(71) Applicant: ExxonMobil Technology and Engineering Company, Spring, TX (US)

(72) Inventor: Deepak MUSALE, Conroe, TX (US)

(21) Appl. No.: 18/056,415

(22) Filed: Nov. 17, 2022

Related U.S. Application Data
(60) Provisional application No. 63/265,419, filed on Dec. 15, 2021.

(51) Int. Cl. A61K 51/12; C02F 9/00; C02F 1/44

(52) U.S. Cl. CPC A61K 51/1282; C02F 9/00; C02F 1/444; C02F 1/441; C02F 1/442; C02F 2101/32

(57) ABSTRACT

Methods of performing targeted alpha therapy of a cancer patient utilizing actinium-225, methods of preparing a targeted alpha therapy drug that includes actinium-225, methods of preparing actinium-225 from radium-226, and methods of recovering radium-226 from an aqueous produced material stream generated from a natural resource extraction process. The methods of recovering radium-226 include separating the radium-226 from the produced material stream to generate recovered radium-226. The methods of preparing actinium-225 include converting the recovered radium-226 into actinium-225. The methods of preparing the targeted alpha therapy drug include incorporating the actinium-225 into the targeted alpha therapy drug. The methods of performing targeted alpha therapy include treating the cancer patient with the targeted alpha therapy drug.

### CROSS-REFERENCE TO RELATED APPLICATION

This application claims the benefit of U.S. Provisional Application Ser. No. 63/265,419, entitled “METHODS OF USING AND CONVERTING RECOVERED RADIUM,” filed Dec. 15, 2021, the disclosure of which is hereby incorporated by reference in its entirety.

### FIELD OF THE DISCLOSURE

The present disclosure relates generally to methods of performing targeted alpha therapy of a cancer patient utilizing actinium-225, methods of preparing a targeted alpha therapy drug that includes actinium-225, methods of preparing actinium-225 from radium-226, and/or methods of recovering radium-226 from an aqueous produced material stream generated from a natural resource extraction process.

### BACKGROUND OF THE DISCLOSURE

Targeted alpha therapy is a promising cancer treatment that incorporates a radioactive isotope, which undergoes alpha decay, into a therapeutic drug. Once provided to a patient, the radioactive isotope undergoes alpha decay, thereby emitting one or more alpha particles. Within the patient, the emitted alpha particles only are able to travel a short distance, generally on the order of a diameter of 2-3 human cells. Thus, and when utilized in conjunction with delivery mechanisms that place the radioactive isotope within close proximity to cancerous cells, the alpha particles may irradiate the cancerous cells while posing only minimal risks to a remainder of the patient's body.

One radioactive isotope, which has shown promise in pre-clinical and clinical studies, is actinium-225. Actinium-225 exhibits several benefits over other available radioactive isotopes. As an example, and upon decay, a total of four alpha particles are emitted from each actinium-225 atom. As another example, actinium-225 has a half-life of 9.9 days, which is sufficient to permit generation of the actinium-225, incorporation into the therapeutic drug, and delivery of the therapeutic drug to the patient. As yet another example, actinium-225 is charged, which may permit attachment of the actinium-225 to antibodies and/or to other cancer-targeting biomolecules, which may permit the actinium-225 to be delivered in a precise, and targeted fashion, to the cancerous cells.

While targeted alpha therapy is a promising cancer treatment, the worldwide supply of applicable radioactive isotopes, such as actinium-225, is many orders of magnitude less than anticipated demand. Thus, there exists a need for improved methods of performing targeted alpha therapy of a cancer patient utilizing actinium-225, methods of preparing a targeted alpha therapy drug that includes actinium-225, methods of preparing actinium-225 from radium-226, and/or methods of recovering radium-226 from a produced material stream generated from a natural resource extraction process.

### SUMMARY OF THE DISCLOSURE

The present disclosure is directed to methods of performing targeted alpha therapy of a cancer patient utilizing actinium-225, methods of preparing a targeted alpha therapy drug that includes actinium-225, methods of preparing actinium-225 from radium-226, and methods of recovering radium-226 from a produced material stream generated from a natural resource extraction process. The methods of recovering radium-226 include providing an aqueous produced material stream, which includes dissolved radium-226 and particulate matter, and filtering the aqueous produced material stream to generate a retentate and an aqueous radium-containing filtrate stream. The retentate includes a major fraction of particulate matter from the aqueous produced material stream. The aqueous radium-containing filtrate stream includes a major fraction of the dissolved radium-226 from the aqueous produced material stream. The methods of recovering radium-226 also include at least partially separating the dissolved radium-226 from a remainder of the aqueous produced material stream, and such separated dissolved radium-226 may be referred to herein as recovered radium-226.

The methods of preparing actinium-225 include converting the recovered radium-226 into actinium-225. The methods of preparing the targeted alpha therapy drug include incorporating the actinium-225 into the targeted alpha therapy drug. The methods of performing targeted alpha therapy include treating the cancer patient with the targeted alpha therapy drug.

### BRIEF DESCRIPTION OF THE DRAWINGS

FIG. 1 is a flowchart depicting examples of methods of preparing actinium-225, methods of preparing a targeted alpha therapy drug, and/or methods of performing targeted alpha therapy, according to the present disclosure.

FIG. 2 is a schematic illustration of examples of a process flow that may be utilized to recover radium-226 from a natural resource extraction process.

FIG. 3 is a flowchart depicting examples of methods of recovering radium-226 from produced material generated from a natural resource extraction process, according to the present disclosure.

### DETAILED DESCRIPTION AND BEST MODE OF THE DISCLOSURE

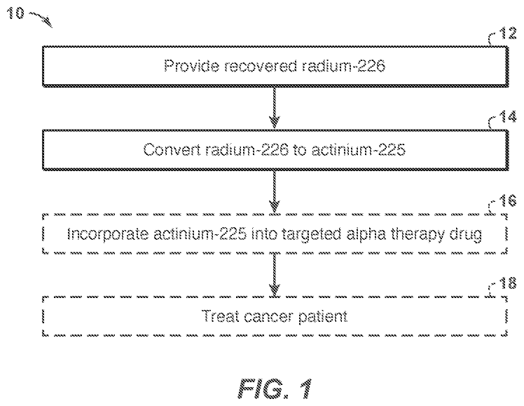

FIGS. 1-3 provide examples of process flows 100 and/or of methods 10 and 200, according to the present disclosure. Elements, components, steps, and/or features that are discussed herein with reference to one or more of FIGS. 1-3 may be included in and/or utilized with any of FIGS. 1-3 without departing from the scope of the present disclosure. In general, elements that are likely to be included in a particular embodiment are illustrated in solid lines, while elements that are optional are illustrated in dashed lines. However, elements that are shown in solid lines may not be essential to all embodiments and, in some embodiments, may be omitted without departing from the scope of the present disclosure.

FIG. 1 is a flowchart depicting examples of methods 10 of preparing actinium-225, methods of preparing a targeted alpha therapy drug, and/or methods of performing targeted alpha therapy, according to the present disclosure. Methods 10 include providing recovered radium-226 at 12 and converting recovered radium-226 to actinium-225 at 14. Methods 10 also may include incorporating the actinium-225 into a targeted alpha therapy drug at 16 and/or treating a cancer patient at 18. When methods 10 include the providing at 12 and the converting at 14, methods 10 may be referred to herein as methods of preparing actinium-225. When methods 10 further include the incorporating at 16, methods 10 may be referred to herein as methods of preparing a targeted alpha therapy drug. When methods 10 further include the treating at 18, methods 10 may be referred to herein as methods of performing targeted alpha therapy treatment of a cancer patient.

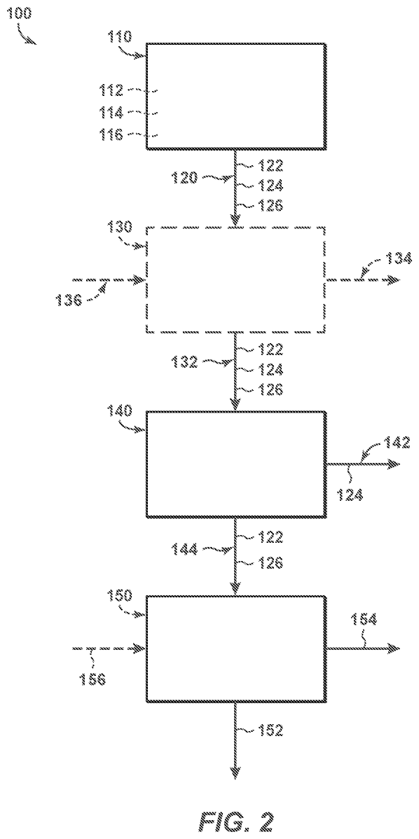

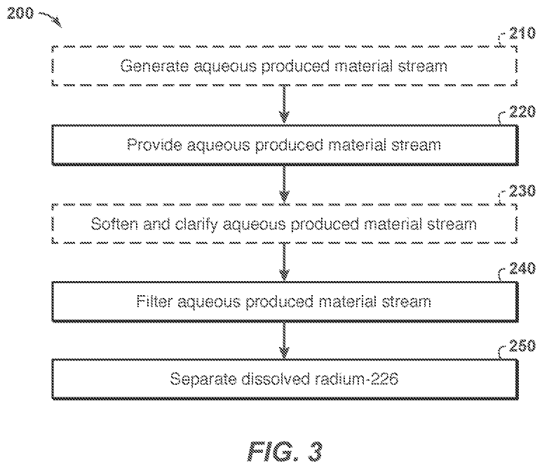

Providing recovered radium-226 at 12 may include providing recovered radium-226 that may be, or may have been, recovered from produced material generated from a natural resource extraction process. In some examples, the produced material may include and/or be an aqueous produced material stream generated by and/or that is a byproduct of the natural resource extraction process. In some examples, the providing at 12 may include recovering the recovered radium-226, the produced material, and/or the aqueous produced material stream from, during, and/or by performing the natural resource extraction process. In some examples, the providing at 12 may include providing the recovered radium-226 utilizing process flows 100 of FIG. 2 and/or by performing methods 200 of FIG. 3.

As discussed, the worldwide supply of radioactive isotopes, which may be utilized within targeted alpha therapy, is many orders of magnitude less than the anticipated demand for such isotopes. As discussed in more detail herein, radium-226 may be converted into actinium-225; and actinium-225 exhibits many characteristics that may be desirable within radioactive isotopes utilized in targeted alpha therapy drugs. With this in mind, the recognition that certain natural resource extraction processes produce product and/or byproduct streams that include significant quantities of radium-226 may permit and/or facilitate increased adoption and/or utilization of targeted alpha therapy in the treatment of cancer patients.

Examples of the natural resource extraction processes include a hydrocarbon extraction process and/or operation, a geothermal extraction process and/or operation, and/or a mining process and/or operation. An example of a produced material that may be produced by such natural resource extraction processes includes process water from hydrocarbon extraction operations. Approximately 300 million barrels of process water are produced globally on a daily basis during hydrocarbon extraction operations. This process water generally has a radium-226 content of 800-7600 pico curie per liter (pCi/L). As such, the process flows and methods, which are disclosed herein, have the potential to revolutionize worldwide cancer treatment options.

However, the worldwide potential for radium-226 recovery from natural resource extraction processes actually is much greater than what may be produced from process water alone. As an example, hydrocarbon extraction operations also generate approximately 230,000 metric tons of sludge each year. This sludge has an average radium-226 concentration of 75 pCi/L. As another example, a given hydrocarbon well can generate approximately 100 metric tons of scale per year. This scale has an average radium-226 concentration of 480 pCi/g. Brines utilized in geothermal operations and/or product and byproduct streams from mining operations also are known to contain recoverable radium-226.

In some examples of methods 10, the providing at 12 may include providing the radium-226 in a form that immediately may be utilized, in the converting at 14, to produce and/or generate actinium-225. However, in other examples, methods 10 and/or the providing at 12 may include performing one or more additional steps prior to performing the converting at 14. As a more specific example, and in some examples of process flows 100 and/or of methods 200, the recovered radium-226 may be provided as a precipitate, as a dry powder, and/or as a concentrated slurry. In some such examples, methods 10 and/or the providing at 12 further may include diluting and/or dissolving the recovered radium-226 prior to performing the converting at 14. In some such examples, the diluting and/or dissolving may include diluting with water and/or dissolving in water.

Converting recovered radium-226 to actinium-225 at 14 may include converting the recovered radium-226 into actinium-225 in any suitable manner and/or utilizing any suitable process. As an example, the converting at 14 may include inducing decay of radium-226 to actinium-225, such as via proton bombardment of the radium-226. Additional examples of processes and/or equipment for the converting at 14 are disclosed in U.S. Patent Application Publication Nos. 2007/0092051 and 2021/0027906, the complete disclosures of which are hereby incorporated by reference. Further examples of processes and/or equipment for the converting at 14 are disclosed in O. D. Maslov, A. V. Sabel'nikov & S. N. Dmitriev, “Preparation of ²²⁵Ac by 226_{Ra}(γ, n) Photonuclear Reaction on an Electron Accelerator, MT-25 Microtron,” Radiochemistry, vol. 48 (2006), pp. 195-197, as well as in PCT Published Patent Application Nos. WO 2020/260210 and WO 2020/254689, and in European Patent Nos. EP0752709 and EP0752710, the complete disclosures of which are hereby incorporated by reference.

Incorporating the actinium-225 into the targeted alpha therapy drug at 16 may include incorporating the actinium-225 into any suitable targeted alpha therapy drug. The targeted alpha therapy drug may be utilized to treat the cancer patient and/or to direct the actinium-225 to cancerous cells in a targeted manner, such as during the treating at 18. As an example, the incorporating at 16 may include combining and/or reacting the actinium-225 with an antibody and/or with a cancer-targeting biomolecule, which may be selected to selectively deliver the actinium-225 to cancerous cells within the patient's body. As discussed in more detail herein, such a configuration may permit and/or facilitate targeted irradiation of the cancerous cells upon decay of the actinium-225 due to the short distances over which alpha particles emitted during the decay of the actinium-225 travel within the patient's body.

Treating the cancer patient at 18 may include treating the patient with the targeted alpha therapy drug. This may include injecting and/or supplying the targeted alpha therapy drug to the patient, or to cancerous cells within the patient, in any suitable manner. Examples of targeted alpha therapy drugs that may utilize actinium-225 and/or of treatments for cancer patients that utilize the targeted alpha therapy drugs are disclosed in Mehran Makvandi, Edouard Dupis, Jonathan W. Engle, F. Meiring Nortier, Michael E. Fassbender, Sam Simon, Eva R. Birnbaum, Robert W.

Atcher, Kevin D. John, Olivier Rixe & Jeffrey P. Norenberg, “Alpha-Emitters and Targeted Alpha Therapy in Oncology: from Basic Science to Clinical Investigations,” Targeted Oncology, vol. 13 (2018), pp. 189-203, and Jean-François Chatal, Françoise Kraeber-Bodéré, Michel Chérel & Ferid Haddad, Alphatherapy, “The New Impetus to Targeted Radionuclide Therapy?,” European Journal of Nuclear Medicine and Molecular Imaging, vol. 45 (2018), pp. 1362-1363.

FIG. 2 is a schematic illustration of examples of a process flow 100 that may be utilized to obtain recovered radium-226 152 from a natural resource extraction process 110. As illustrated in FIG. 2, natural resource extraction process 110 produces and/or generates an aqueous produced material stream 120. Aqueous produced material stream 120 includes dissolved radium-226 122, particulate matter 124, and water 126. Examples of natural resource extraction process 110 include a hydrocarbon extraction process 112, a geothermal extraction process 114, and/or a mining process 116.

In some examples, and as illustrated in dashed lines in FIG. 2, aqueous produced material stream 120 may be provided to a softening and clarifying structure 130. Within softening and clarifying structure 130, aqueous produced material stream 120 may be combined with a softening and clarifying material 136, such as to produce and/or generate a calcium and/or magnesium stream 134 and a softened and clarified aqueous produced material stream 132, which includes dissolved radium-226 122, particulate matter 124, and water 126 but may have a lower concentration of calcium and/or magnesium when compared to aqueous produced material stream 120. Examples of softening and clarifying structure 130, of equipment that may form a portion of softening and clarifying structure 130, and/or of processes that may be performed within softening and clarifying structure 130 are disclosed in U.S. Patent Application Publication Nos. 2021/0163327 and 2017/0247270, the complete disclosures of which are hereby incorporated by reference.

In examples of process flow 100 that include softening and clarifying structure 130, softened and clarified aqueous produced material stream 132 then is provided to a filter structure 140. Alternatively, and in examples of process flow 100 that do not include softening and clarifying structure 130, aqueous produced material stream 120 is directly provided to filter structure 140.

Filter structure 140 may receive aqueous produced material stream 120 or softened and clarified aqueous produced material stream 132 and may separate the corresponding stream into a retentate 142 and an aqueous radium-containing filtrate stream 144. Retentate 142, which also may be referred to herein as a periodically or continuously produced retentate stream 142, may include particulate matter 124, at least a fraction of particulate matter 124, and/or a major fraction of particulate matter 124 from aqueous produced material stream 120. Aqueous radium-containing filtrate stream 144 includes dissolved radium-226 122 from aqueous produced material stream 120, which may be dissolved in water 126. Stated differently, filter structure 140 may be utilized to separate particulate contaminates, such as may be defined by particulate matter 124, from a remainder of aqueous produced material stream 120. Examples of filter structure 140 include microfiltration structures and/or ultrafiltration structures. As used herein, a “major fraction” refers to the largest, or majority, fraction of the corresponding substance, with the one or more other fractions optionally being referred to as minor or minority fractions.

Aqueous radium-containing filtrate stream 144 then is provided to a separation structure 150. Separation structure 150 separates recovered radium-226 152 from a remainder 154 of aqueous radium-containing filtrate stream 144. In some examples, this separation may be accomplished via combination of aqueous radium-containing filtrate stream 144 with a separation-enhancing material 156.

FIG. 3 is a flowchart depicting examples of methods 200 of recovering radium-226 from produced material generated from a natural resource extraction process, according to the present disclosure, such as natural resource extraction process 110 of FIG. 2. Methods 200 may include generating an aqueous produced material stream at 210, and methods 200 include providing the aqueous produced material stream at 220. Methods 200 also may include softening and clarifying the aqueous produced material stream at 230, and methods 200 include filtering the aqueous produced material stream at 240 and separating dissolved radium-226 at 250.

Generating the aqueous produced material stream at 210 may include generating the aqueous produced material stream from the natural resource extraction process. This may include performing the natural resource extraction process prior to and/or at least partially concurrently with the providing at 220, the softening and clarifying at 230, the filtering at 240, and/or the separating at 250.

Providing the aqueous produced material stream at 220 may include providing any suitable aqueous produced material stream that includes radium-226, particulate matter, and water. In some examples, the providing at 220 may include providing a produced water stream, which was produced from the natural resource extraction process. In some such examples, the generating at 210 may include producing the produced water stream from the natural resource extraction process. Examples of the produced water stream include flow-back water from production of oil, natural gas, coal bed methane, gas condensate, and/or natural gas liquids. In some such examples, the providing at 220 further may include de-oiling this produced water stream prior to utilization of the produced water stream during the softening and clarifying at 230 and/or the filtering at 240. It is within the scope of the present disclosure that the produced water stream may have a radium-226 concentration of 1 pCi/L to 20,000 pCi/L.

In some such examples, the produced water stream may include and/or be a byproduct stream from the natural resource extraction process. In a specific example, the natural resource extraction process may include a hydrocarbon well configured to produce a fluid hydrocarbon stream from a subsurface region. In some such examples, the produced water stream may include water produced from the subsurface region via the hydrocarbon well. In some such examples, the generating at 210 may include producing the produced water stream from the subsurface region.

In some examples, the natural resource extraction process may include and/or be a geothermal process, which may be performed within a geothermal formation. In some such examples, the produced water stream may include and/or be a geothermal brine, which previously was circulated through the geothermal formation. In some such examples, the providing at 220 may include circulating the geothermal brine through the geothermal formation.

In some examples, the natural resource extraction process may include and/or be a mining process, which may be performed within and/or utilizing a mine. In some such examples, the produced water stream may include and/or be water recovered from the mine. In some such examples, the generating at 210 may include recovering the water from the mine. Examples of the mine include a metal mine and a coal mine.

In some examples, the providing the aqueous produced material stream may include incorporating byproduct from the natural resource extraction process into a water stream to produce and/or generate the aqueous produced material stream. Examples of the byproduct include a natural gas liquid, a gas condensate, a sludge, and/or a scale. The sludge may be derived and/or collected from water storage ponds, three-phase separators, processing equipment, or gas transfer pipelines, for example; and may have a radium-226 concentration of 1 pCi/gram of sludge to 40,000 pCi/gram of sludge. The scale may be derived and/or collected from surfaces of process water transfer pipelines, equipment surfaces, production tubing, sucker rods, heat exchangers, filters, and the like; and may have a radium-226 concentration of 1 pCi/gram of scale to 400,000 pCi/gram of scale.

Softening and clarifying the aqueous produced material stream at 230 may include softening and clarifying the aqueous produced material stream subsequent to the providing at 220 and/or prior to the filtering at 240. This may include softening and clarifying the aqueous produced material stream to produce and/or generate a softened and clarified aqueous produced material stream. In a specific example, the softening and clarifying at 230 may include lime softening the aqueous produced material stream, via addition of lime to the aqueous produced material stream, to generate the softened and clarified aqueous produced material stream. Additionally or alternatively, the softening and clarifying at 230 may include removing calcium and/or magnesium from the aqueous produced material stream. Additional examples of the softening and clarifying at 230 are disclosed in U.S. Patent Application Publication No. 2017/0247270, the complete disclosure of which is hereby incorporated by reference.

Filtering the aqueous produced material stream at 240 may include filtering the aqueous produced material stream to produce and/or generate a retentate and an aqueous radium-containing filtrate stream. The retentate includes a major fraction of the particulate matter from the aqueous produced material stream, while the aqueous radium-containing filtrate stream includes a major fraction of the dissolved radium-226 from the aqueous produced material stream. The filtering at 240 may include filtering in any suitable manner. As examples, the filtering at 240 may include utilizing microfiltration and/or ultrafiltration to separate the retentate from the aqueous radium-containing filtrate stream. If microfiltration is utilized, a pore size of microfiltration equipment may be 0.1 micrometers to 10 micrometers. If ultrafiltration is utilized, a pore size of ultrafiltration equipment may be 0.01 micrometers to 0.1 micrometers. In some examples, the filtering at 240 may include utilizing a filter membrane. The filter membrane may be organic, inorganic, and/or a combination of organic and inorganic components. The filter membrane may be utilized in a submerged mode and/or a pressurized mode. The filter membrane may be utilized in a hollow fiber, multi-bore, spiral-wound, tubular, and/or flat-sheet configuration.

Separating dissolved radium-226 at 250 may include at least partially separating, isolating, dividing, and/or segregating the dissolved radium-226 from a remainder of the aqueous radium-containing filtrate stream, such as to produce and/or generate recovered radium-226. This may include separating the dissolved radium-226 in any suitable manner and/or utilizing any suitable process, including those that are discussed in more detail herein. In general, the recovered radium-226, as produced during the separating at 250, may be more concentrated and/or more pure when compared to dissolved radium-226 in the aqueous radium-containing filtrate stream.

The prior discussion of methods 200 applies generally to recovery of radium-226 from a variety of different aqueous produced material streams, which may be generated from a variety of different natural resource extraction processes. The following are more specific examples of methods 200 that may be performed for specific aqueous produced material streams generated from specific natural resource extraction processes.

In a specific example, the aqueous produced material stream includes produced water, which is produced from a natural resource extraction process, such as hydrocarbon extraction via a hydrocarbon well. This example includes performing the providing at 220, the filtering at 240, and the separating at 250. In this example, the filtering at 240 includes utilizing ultrafiltration to separate the retentate from the aqueous radium-containing filtrate stream.

Also in this example, the separating at 250 includes precipitating dissolved radium-226 from the aqueous radium-containing filtrate stream, such as to generate a radium-containing precipitate suspended in water. The separating at 250 further may include concentrating the radium-containing precipitate as the recovered radium-226. Examples of precipitation processes that may be utilized to form the radium-containing precipitate are disclosed in U.S. Patent Application Publication Nos. 2021/0163327 and 2017/0217802, as well as in U.S. Pat. No. 8,894,864, the complete disclosures of which are hereby incorporated by reference.

The precipitating includes combining the aqueous radium-containing filtrate stream with a precipitant, which includes barium and/or sulfate. The radium-containing precipitate includes radium sulfate and/or radium-barium sulfate, which include radium-226. Barium sulfate also may be included within the radium-containing precipitate. More specific examples of the precipitant include barium chloride dehydrate, barium nitrate, ammonium sulfate, sulfuric acid, sodium sulfate, and/or potassium sulfate. In a specific example, the precipitating may include saturating the water with barium sulfate.

Also in this example, the concentrating includes at least partially, or even substantially, dewatering the radium-containing precipitate to form an aqueous slurry of the radium-containing precipitate that includes the recovered radium-226. The dewatering may include utilizing ultrafiltration, nanofiltration, reverse osmosis, evaporation, and/or centrifugation to at least partially separate the radium-containing precipitate from water in the aqueous slurry of the radium-containing precipitate.

Also in this example, and subsequent to the filtering at 240 and prior to the precipitating, the at least partially separating at 250 may include concentrating the aqueous radium-containing filtrate stream, such as via nanofiltering the aqueous radium-containing filtrate stream and/or via evaporation of water from the aqueous radium-containing filtrate stream. In such examples, the aqueous radium-containing filtrate stream may include at least one low-solubility metal-containing compound, and the concentrating the aqueous radium-containing filtrate stream may include concentrating to less than a saturation concentration of the at least one low-solubility metal-containing compound. Examples of the at least one low-solubility metal-containing compound include strontium sulfate, calcium carbonate, iron carbonate, calcium sulfate, and/or calcium phosphate.

In another specific example, the aqueous produced material stream includes produced water, which is produced from a natural resource extraction process, such as hydrocarbon extraction via a hydrocarbon well. This example includes performing the providing at 220, the filtering at 240, and the separating at 250. In this example, the filtering at 240 includes utilizing ultrafiltration to separate the retentate from the aqueous radium-containing filtrate stream.

Also in this example, the separating at 250 includes precipitating dissolved radium-226 from the aqueous radium-containing filtrate stream, such as to generate a radium-containing precipitate suspended in water. The separating at 250 further may include concentrating the radium-containing precipitate as the recovered radium-226. Examples of precipitation processes that may be utilized to form the radium-containing precipitate are disclosed in U.S. Patent Application Publication Nos. 2021/0163327 and 2017/0217802, as well as in U.S. Pat. No. 8,894,864, the complete disclosures of which are hereby incorporated by reference.

The precipitating includes combining the aqueous radium-containing filtrate stream with a precipitant, which includes phosphate. The radium-containing precipitate includes calcium phosphate that incorporates radium-226. More specific examples of the precipitant include a water-soluble phosphate compound, such as a phosphate salt, sodium phosphate, potassium phosphate, ammonium phosphate, and phosphoric acid.

Also in this example, the concentrating includes at least partially, or even substantially, dewatering the radium-containing precipitate to form an aqueous slurry of the radium-containing precipitate that includes recovered radium-226. The dewatering may include and/or be a single-step or a multi-step concentrating process that may be performed utilizing ultrafiltration, nanofiltration, reverse osmosis, evaporation, and/or centrifugation to at least partially separate the radium-containing precipitate from water in the aqueous slurry of the radium-containing precipitate.

Also in this example, and subsequent to the filtering at 240 and prior to the precipitating, the at least partially separating at 250 further may include combining the aqueous radium-containing filtrate stream with barium and/or sulfate. The barium may be derived from barium chloride dehydrate, and/or barium nitrate. The sulfate may be derived from ammonium sulfate, sulfuric acid, sodium sulfate, and/or potassium sulfate.

In another specific example, the aqueous produced material stream includes produced water, which is produced from a natural resource extraction process, such as hydrocarbon extraction via a hydrocarbon well. This example includes performing the providing at 220, the softening and clarifying at 230, the filtering at 240, and the separating at 250. In this example, the filtering at 240 includes utilizing ultrafiltration to separate the retentate from the aqueous radium-containing filtrate stream.

Also in this example, the separating at 250 includes adjusting a pH of the aqueous radium-containing filtrate stream to between 6 and 9 to generate a pH-adjusted filtrate stream and contacting the pH-adjusted filtrate stream with a metal-organic framework adsorbent to adsorb the dissolved radium-226 onto the metal-organic framework adsorbent as the recovered radium-226. The metal-organic framework adsorbent may include nanoporous materials that may be assembled with functional organic linkers and metal ions and/or cluster nodes. The metal-organic framework adsorbent may be anionic and neutral, such as to facilitate adsorption of cationic radium-226 ions. The metal-organic framework adsorbent may be selected to define a pore size that corresponds to a diameter of dissolved radium-226 ions. Examples of the pore size include pores sizes of at least 2.8 Angstroms and at most 3.2 Angstroms.

In another specific example, the aqueous produced material stream includes produced water, which is produced from a natural resource extraction process, such as hydrocarbon extraction via a hydrocarbon well. This example includes performing the providing at 220, the softening and clarifying at 230, the filtering at 240, and the separating at 250. In this example, the filtering at 240 includes utilizing ultrafiltration to separate the retentate from the aqueous radium-containing filtrate stream.

Also in this example, the separating at 250 includes adjusting a pH of the aqueous radium-containing filtrate stream to between 6 and 9 to generate a pH-adjusted filtrate stream, contacting the pH-adjusted filtrate stream with a surfactant to generate radium-containing micelles, and separating the radium-containing micelles from the pH-adjusted filtrate stream as the recovered radium. Examples of the surfactant include a cationic surfactant, an anionic surfactant, a zwitterionic surfactant, and/or a non-ionic surfactant.

The contacting the pH-adjusted filtrate stream with the surfactant may include mixing the surfactant into the pH-adjusted filtrate stream to a surfactant concentration that is greater than a critical micelle concentration of the surfactant within the pH-adjusted filtrate stream. The separating the radium-containing micelles from the pH-adjusted filtrate stream may include filtering the radium-containing micelles from the pH-adjusted filtrate stream, such as via utilizing ultrafiltration and/or nanofiltration to separate the radium-containing micelles from the pH-adjusted filtrate stream as a radium-containing micelle retentate.

In another specific example, the aqueous produced material stream includes produced water, which is produced from a natural resource extraction process, such as hydrocarbon extraction via a hydrocarbon well. This example includes performing the providing at 220, the softening and clarifying at 230, the filtering at 240, and the separating at 250. In this example, the filtering at 240 includes utilizing ultrafiltration to separate the retentate from the aqueous radium-containing filtrate stream.

Also in this example, the separating at 250 includes utilizing an electrodialysis process and/or an electodialysis reversal process that includes a bivalent cation selective ion exchange membrane to concentrate bivalent cations, such as radium-226 cations, in water. Also in this example, the separating at 250 further includes combining the concentrated bivalent cations with sulfate to precipitate radium sulfate and separating the precipitated radium sulfate from the aqueous radium-containing filtrate stream. The separating the precipitated radium sulfate from the aqueous radium-containing filtrate stream may include filtering the precipitated radium sulfate from the aqueous radium-containing filtrate stream, such as utilizing ultrafiltration.

In another specific example, the aqueous produced material stream includes water within which scale has been incorporated and/or at least partially dissolved. This scale may be recovered from the natural resource extraction process, such as via being scraped from oil and gas production equipment, and mixed with the water. This example includes performing the providing at 220, the filtering at 240, and the separating at 250.

In this example, the providing at 220 includes washing the scale, which previously was recovered from deposits within the hydrocarbon production equipment, with an aqueous wash solution. The aqueous wash solution includes a chelating agent and has a pH that is above a pKa of the chelating agent, such that radium-226 complexes with the chelating agent to generate a radium-containing aqueous wash solution that defines the aqueous produced material stream. Examples of the chelating agent include ethylenediaminetetraacetic acid (EDTA), nitrilotriacetic acid (NTA), citric acid, tetraxetan (DOTA), picolinic acid, and an 18-membered bis-picolinate diazacrown ring (MACROPA).

Also in this example, the separating at 250 includes concentrating the aqueous radium-containing filtrate stream. The concentrating may include concentrating via nanofiltration, reverse osmosis, and/or evaporation of the aqueous radium-containing filtrate stream to generate a concentrated aqueous radium-containing stream.

The separating at 250 also may include reducing a pH of the concentrated aqueous radium-containing stream to below the pKa of the chelating agent to de-complex radium-226 and the chelating agent. The separating at 250 subsequently may include separating the radium-226 from the chelating agent via ultrafiltration to generate a concentrated radium-containing filtrate stream and a chelating agent stream. The separating at 250 subsequently may include further concentrating the concentrated radium-containing filtrate stream utilizing nanofiltration and/or reverse osmosis and utilizing the chelating agent stream for at least a fraction of the chelating agent utilized during the washing.

In the present disclosure, several of the illustrative, non-exclusive examples have been discussed and/or presented in the context of flow diagrams, or flow charts, in which the methods are shown and described as a series of blocks, or steps. Unless specifically set forth in the accompanying description, it is within the scope of the present disclosure that the order of the blocks may vary from the illustrated order in the flow diagram, including with two or more of the blocks (or steps) occurring in a different order and/or concurrently.

As used herein, the term “and/or” placed between a first entity and a second entity means one of (1) the first entity, (2) the second entity, and (3) the first entity and the second entity. Multiple entities listed with “and/or” should be construed in the same manner, i.e., “one or more” of the entities so conjoined. Other entities may optionally be present other than the entities specifically identified by the “and/or” clause, whether related or unrelated to those entities specifically identified. Thus, as a non-limiting example, a reference to “A and/or B,” when used in conjunction with open-ended language such as “comprising” may refer, in one embodiment, to A only (optionally including entities other than B); in another embodiment, to B only (optionally including entities other than A); in yet another embodiment, to both A and B (optionally including other entities). These entities may refer to elements, actions, structures, steps, operations, values, and the like.

As used herein, the phrase “at least one,” in reference to a list of one or more entities should be understood to mean at least one entity selected from any one or more of the entities in the list of entities, but not necessarily including at least one of each and every entity specifically listed within the list of entities and not excluding any combinations of entities in the list of entities. This definition also allows that entities may optionally be present other than the entities specifically identified within the list of entities to which the phrase “at least one” refers, whether related or unrelated to those entities specifically identified. Thus, as a non-limiting example, “at least one of A and B” (or, equivalently, “at least one of A or B,” or, equivalently “at least one of A and/or B”) may refer, in one embodiment, to at least one, optionally including more than one, A, with no B present (and optionally including entities other than B); in another embodiment, to at least one, optionally including more than one, B, with no A present (and optionally including entities other than A); in yet another embodiment, to at least one, optionally including more than one, A, and at least one, optionally including more than one, B (and optionally including other entities). In other words, the phrases “at least one,” “one or more,” and “and/or” are open-ended expressions that are both conjunctive and disjunctive in operation. For example, each of the expressions “at least one of A, B, and C,” “at least one of A, B, or C,” “one or more of A, B, and C,” “one or more of A, B, or C,” and “A, B, and/or C” may mean A alone, B alone, C alone, A and B together, A and C together, B and C together, A, B, and C together, and optionally any of the above in combination with at least one other entity.

In the event that any patents, patent applications, or other references are incorporated by reference herein and (1) define a term in a manner that is inconsistent with and/or (2) are otherwise inconsistent with, either the non-incorporated portion of the present disclosure or any of the other incorporated references, the non-incorporated portion of the present disclosure shall control, and the term or incorporated disclosure therein shall only control with respect to the reference in which the term is defined and/or the incorporated disclosure was present originally.

As used herein the terms “adapted” and “configured” mean that the element, component, or other subject matter is designed and/or intended to perform a given function. Thus, the use of the terms “adapted” and “configured” should not be construed to mean that a given element, component, or other subject matter is simply “capable of” performing a given function but that the element, component, and/or other subject matter is specifically selected, created, implemented, utilized, programmed, and/or designed for the purpose of performing the function. It is also within the scope of the present disclosure that elements, components, and/or other recited subject matter that is recited as being adapted to perform a particular function may additionally or alternatively be described as being configured to perform that function, and vice versa.

As used herein, the phrase, “for example,” the phrase, “as an example,” and/or simply the term “example,” when used with reference to one or more components, features, details, structures, embodiments, and/or methods according to the present disclosure, are intended to convey that the described component, feature, detail, structure, embodiment, and/or method is an illustrative, non-exclusive example of components, features, details, structures, embodiments, and/or methods according to the present disclosure. Thus, the described component, feature, detail, structure, embodiment, and/or method is not intended to be limiting, required, or exclusive/exhaustive; and other components, features, details, structures, embodiments, and/or methods, including structurally and/or functionally similar and/or equivalent components, features, details, structures, embodiments, and/or methods, are also within the scope of the present disclosure.

As used herein, “at least substantially,” when modifying a degree or relationship, may include not only the recited “substantial” degree or relationship, but also the full extent of the recited degree or relationship. A substantial amount of a recited degree or relationship may include at least 75% of the recited degree or relationship. For example, an object that is at least substantially formed from a material includes objects for which at least 75% of the objects are formed from the material and also includes objects that are completely formed from the material. As another example, a first length that is at least substantially as long as a second length includes first lengths that are within 75% of the second length and also includes first lengths that are as long as the second length.

### INDUSTRIAL APPLICABILITY

The systems and methods disclosed herein are applicable to the cancer treatment and natural resource extraction industries.

It is believed that the disclosure set forth above encompasses multiple distinct inventions with independent utility. While each of these inventions has been disclosed in its preferred form, the specific embodiments thereof as disclosed and illustrated herein are not to be considered in a limiting sense as numerous variations are possible. The subject matter of the inventions includes all novel and non-obvious combinations and subcombinations of the various elements, features, functions, and/or properties disclosed herein. Similarly, where the claims recite “a” or “a first” element or the equivalent thereof, such claims should be understood to include incorporation of one or more such elements, neither requiring nor excluding two or more such elements.

It is believed that the following claims particularly point out certain combinations and subcombinations that are directed to one of the disclosed inventions and are novel and non-obvious. Inventions embodied in other combinations and subcombinations of features, functions, elements, and/or properties may be claimed through amendment of the present claims or presentation of new claims in this or a related application. Such amended or new claims, whether they are directed to a different invention or directed to the same invention, whether different, broader, narrower, or equal in scope to the original claims, are also regarded as included within the subject matter of the inventions of the present disclosure.

1. A method of performing targeted alpha therapy of a cancer patient, the method comprising:
providing recovered radium-226, which was recovered from produced material generated from a hydrocarbon extraction process; and
converting the recovered radium-226 into actinium-225;
incorporating the actinium-225 into a targeted alpha therapy drug; and
treating the cancer patient with the targeted alpha therapy drug.

2. A method of recovering radium-226 from produced material generated from a natural resource extraction process, the method comprising:
providing an aqueous produced material stream that includes dissolved radium-226 and particulate matter;
filtering the aqueous produced material stream to generate a retentate, which includes a major fraction of the particulate matter, and an aqueous radium-containing filtrate stream, which includes a major fraction of the dissolved radium-226; and
at least partially separating the dissolved radium-226, as recovered radium-226, from a remainder of the aqueous radium-containing filtrate stream.

3. The method of claim 2, wherein the providing the aqueous produced material stream includes providing a produced water stream produced from the natural resource extraction process.

4. The method of claim 3, wherein the method further includes producing the produced water stream from the natural resource extraction process.

5. The method of claim 3, wherein the produced water stream is a byproduct stream of the natural resource extraction process.

6. The method of claim 3, wherein the natural resource extraction process includes a hydrocarbon well configured to produce a fluid hydrocarbon stream from a subsurface region, and further wherein the produced water stream includes water produced from the subsurface region via the hydrocarbon well.

7. The method of claim 6, wherein, prior to the providing, the method further includes producing the water from the subsurface region.

8. The method of claim 3, wherein the natural resource extraction process includes a geothermal formation, and further wherein the produced water stream includes a geothermal brine that previously was circulated through the geothermal formation.

9. The method of claim 8, wherein, prior to the providing, the method further includes circulating the geothermal brine through the geothermal formation.

10. The method of claim 3, wherein the natural resource extraction process includes a mining process performed within a mine, and further wherein the produced water stream includes water recovered from the mine.

11. The method of claim 10, wherein, prior to the providing, the method further includes recovering the water from the mine.

12. The method of claim 10, wherein the mine includes at least one of a metal mine and a coal mine.

13. The method of claim 2, wherein the providing the aqueous produced material stream includes incorporating a byproduct from the natural resource extraction process into a water stream to generate the aqueous produced material stream.

14. The method of claim 13, wherein the byproduct includes at least one of:
(i) a natural gas liquid;
(ii) a gas condensate;
(iii) a sludge; and
(iv) a scale.

15. The method of claim 2, wherein the at least partially separating includes:
(i) precipitating the dissolved radium-226 from the aqueous radium-containing filtrate stream to generate a radium-containing precipitate suspended in water; and
(ii) concentrating the radium-containing precipitate.

16. The method of claim 15, wherein:
(i) the filtering includes utilizing ultrafiltration to separate the retentate from the aqueous radium-containing filtrate stream;
(ii) the precipitating includes combining the aqueous radium-containing filtrate stream with a precipitant, which includes at least one of barium and sulfate, to precipitate at least one of radium sulfate, barium sulfate, and radium-barium sulfate as the radium-containing precipitate; and
(iii) the concentrating includes partially dewatering the radium-containing precipitate to form an aqueous slurry of the radium-containing precipitate, wherein the aqueous slurry of the radium-containing precipitate includes the recovered radium-226.

17. The method of claim 16, wherein the concentrating includes dewatering the radium-containing precipitate.

18. The method of claim 16, wherein the concentrating includes at least one of:
(i) utilizing ultrafiltration to at least partially separate the radium-containing precipitate from water in the aqueous slurry of the radium-containing precipitate;
(ii) utilizing nanofiltration to at least partially separate the radium-containing precipitate from water in the aqueous slurry of the radium-containing precipitate;
(iii) utilizing reverse osmosis to at least partially separate the radium-containing precipitate from water in the aqueous slurry of the radium-containing precipitate;
(iv) utilizing evaporation to at least partially separate the radium-containing precipitate from water in the aqueous slurry of the radium-containing precipitate; and
(v) utilizing centrifugation to at least partially separate the radium-containing precipitate from water in the aqueous slurry of the radium-containing precipitate.

19. The method of claim 16, wherein, subsequent to the filtering and prior to the precipitating, the at least partially separating further includes concentrating the aqueous radium-containing filtrate stream.

20. The method of claim 19, wherein the concentrating the aqueous radium-containing filtrate stream includes at least one of:
(i) nanofiltering the aqueous radium-containing filtrate stream; and
(ii) evaporating water from the aqueous radium-containing filtrate stream.

21-23. (canceled)
